(19) United States
(12) Patent Application Publication
(10) Pub. No.: US 2016/0317408 A1
(43) Pub. Date: Nov. 3, 2016

(54) KERATIN FIBRE DYEING METHOD

(71) Applicant: ALFA PARF GROUP S.p.A., Bologna (IT)

(72) Inventors: Katiuscia Grevalcuore, Bergamo (IT); Antonio Consoli, Urgnano (IT); Luca Carminati, Curno (IT); Massimo Fabbi, Mozzo (IT)

(21) Appl. No.: 15/136,264

(22) Filed: Apr. 22, 2016

(30) Foreign Application Priority Data
Apr. 30, 2015 (IT) 102015000013671

(51) Int. Cl. A61K 8/34; A61Q 5/10; A61K 8/49; A61K 8/22; A61K 8/41

(52) U.S. Cl. CPC A61K 8/347; A61K 8/22; A61K 8/415; A61K 8/4926; A61Q 5/10; A61K 2800/4324

(57) ABSTRACT

The present invention relates to methods of dyeing the hair with oxidative dyes devoid of p-ph enylenediamine and p-toluenediamine and containing N,N-bis(2-hydroxyethyl)-p-phenylenediamine, p-aminophenol, 4-amino-m-cresol, hydroxyethyl-p-phenylenediamine, 1-hydroxyethyl-4,5-diamino pyrazole, 4-chlorine-resorcinol, 2-methylresorcinol, 2,4-diaminophenoxyethanol and 2-amino-3-hydroxypyridine, which possess an improved sensitising power and can be used in mixtures of the various shades with predictable results.

This application is a U.S. Non-provisional application which claims priority to and the benefit of Italian patent application no. 102015000013671 filed on Apr. 30 2015, the content of which is incorporated herein by reference in its entirety.

The present invention relates to a hair dyeing method using oxidative dyes devoid of p-phenylenediamine and p-toluenediamine, which contain an activator and dyes selected from N,N-bis(2-hydroxyethyl)-p-phenylenediamine, p-aminophenol, 4-amino-m-cresol, hydroxyethyl-p-phenylenediamine, 1-hydroxyethyl-4,5-diamino pyrazole, 4-chlororesorcinol, 2-methylresorcinol, 2,4-diaminophenoxyethanol and 2-amino-3-hydroxypyridine. The method according to the invention reduces the sensitising power, and the compositions can be used in mixtures of the different shades with predictable results.

### PRIOR ART

The desire to change the colour of natural and synthetic fibres has developed over the years. In particular, hair dyeing in humans is motivated by changes in style and fashion. There are currently numerous trends in hair dyeing. In the past, the hair was mainly dyed to conceal areas of grey hair, whereas there is now increased demand to have a hair colour based on fashion as an expression of the personality. Two main hair dyeing methods are widely used. One is the semi-permanent system, which involves colouring the hair with products containing direct non-oxidative dyes. Although both the colouring capacity of the dyes used for this purpose and their ability to remain on the hair as long as possible are optimised, the shade of colour gradually fades with each wash. Depending on the product used and the hair type, these dyes do not generally last for more than 10 washes. Although they are designed for direct dyeing, some of these dyes can also be applied in the presence of hydrogen peroxide to intensify the resulting shade and obtain better uniformity from root to tip. In addition to the semi-permanent system, oxidative dyes have achieved considerable importance in cosmetics in the field of conventional hair dyes. The colour is created by the reaction of primary intermediates and couplers in the presence of an oxidant. In addition to the creation of colour effects, very strict requirements are imposed for oxidative dyes designed for human hair treatment. Firstly, the dyes must be harmless and possibly non-sensitising. It must also be possible to produce a wide range of different shades by combining primary intermediates and suitable couplers. Moreover, the hair dyes produced must possess good resistance to washing, light, sweat, permanent wave treatments, acids, bases and abrasions. In any event said hair dyes, once applied, should usually remain stable for four to six weeks under normal conditions. The oxidative system is based on the reaction of primary intermediates with couplers; both types of molecule are practically colourless. In the presence of air or oxidants such as hydrogen peroxide, primary aromatic amines with a hydroxyl or additional amine group, substituted or not substituted, in the para or ortho position, react with resorcinol, m-aminophenol, m-phenylenediamine or 1-naphthol couplers.

As the size of the dye molecules formed in the cuticle is larger than the size of the starting primary intermediates and of the highly diffusible couplers, no significant fading takes place after dyeing. An oxidative hair dye that produces a long-lasting shade is therefore also called “permanent” system.

The primary dyes generally used in the cosmetic field for manufacturing oxidative hair dyes are p-phenylenediamine and p-toluenediamine.

Opinion 1509/13 of the Scientific Committee on Consumer Safety (SCCS) entitled “Memorandum on hair dye chemical sensitisation”, dated Feb. 26 2013, lists the sensitising power values of various oxidative and direct dyes. In particular they are classified, according to a criterion described in the document, as Extreme Sensitisers, Strong Sensitisers, Moderate Sensitisers, Insufficiently Tested Dyes, and Non-classifiable Dyes. The dyes p-phenylenediamine and p-toluenediamine are both classified as “Extreme”. Other primary dyes, such as hydroxyethyl-p-phenylenediamine, N,N-bis(2-hydroxyethyl)-p-phenylenediamine, 1-hydroxyethyl-4,5-diamino pyrazole, resorcinol, 4-amino-m-cresol and m-aminophenol, possess a lower sensitising power and are classified as “Strong”, while 2-methylresorcinol, 4-chlororesorcinol and 2,4-diaminophenoxyethanol are classified as “Moderate”. 2-Amino-3-hydroxypyridine is classified as “not classifiable”. P-aminophenol is classified as a “strong” sensitiser in Opinion SCCS/1409/11. One of the couplers most commonly used is m-aminophenol which, combined with p-phenylenediamine and p-toluenediamine, gives violet-brown colours, and is therefore widely used for natural, gold and copper dyes.

U.S. Pat. No. 3,884,627 describes the dye N,N-bis(2-hydroxyethyl)-p-phenylene-diamine, which is characterised by a better toxicological profile than the molecules p-phenylenediamine and p-toluenediamine. Said patent also discloses the combination of N,N-bis(2-hydroxyethyl)-p-phenylenediamine with meta-aminophenol which, unlike the other primary dyes described above, gives a blue colour. This result is a problem for the formulation of shades, because it makes it impossible to use m-aminophenol to obtain a brown colour.

The purpose of the invention is to develop a composition for dyeing keratin, in particular hair, which guarantees a better sensitising power than the use of p-phenylenediamine or p-toluenediamine and a predictable result in the development of the colour when the various shades are intermixed.

### SUMMARY OF THE INVENTION

It has been found that said purposes are achieved by oxidative compositions comprising an activator, at least one primary dye selected from N,N-bis(2-hydroxyethyl)-p-phenylenediamine, p-aminophenol, 4-amino-m-cresol, hydroxy-ethyl-p-phenylenediamine and 1-hydroxyethyl-4,5-diamino pyrazole and at least one dye coupler selected from 4-chlororesorcinol, 2-methylresorcinol, 2,4-diaminophenoxyethanol and 2-amino-3-hydroxypyridine, said compositions being characterised by the absence of p-phenylenediamine, p-toluenediamine and m-aminophenol. The invention achieves the aim of reducing the sensitising power of the oxidative compositions normally used in hair dye.

Compositions containing an activator, at least one primary dye selected from N,N-bis(2-hydroxyethyl)-p-phenylenediamine and p-aminophenol, hydroxyethyl-p-phenylenediamine, 4-amino-m-cresol and at least one dye coupler selected from 4-chlororesorcinol, 2-methylresorcinol and 2,4-diaminophenoxyethanol, wherein said compositions do not contain p-phenylenediamine, p-toluenediamine, m-aminophenol or resorcinol, are novel, and are a further object of the invention.

### DETAILED DESCRIPTION OF THE INVENTION

The method according to the invention comprises the application to the hair of compositions containing an activator, at least one primary dye selected from N,N-bis(2-hydroxyethyl)-p-phenylenediamine, p-aminophenol, 4-amino-m-cresol, hydroxyethyl-p-phenylenediamine and 1-hydroxyethyl-4,5-diamino pyrazole and at least one dye coupler selected from 4-chlororesorcinol, 2-methylresorcinol, 2,4-diaminophenoxyethanol and 2-amino-3-hydroxypyridine, said compositions being characterised by the absence of p-phenylenediamine, p-toluenediamine m-aminophenol and resorcinol, and can take the form of O/W (oil-in-water) or W/O (water-in-oil) emulsions, liquids, two-phase liquids, gels or oils.

Despite the absence of the primary dyes p-phenylenediamine and p-toluenediamine, the invention enables the formulator to create the shades and levels available from the hairdresser, and in particular the natural, gold, ash, violet, copper, pearly, mahogany, red and khaki (green) series, super-lighteners and intensifiers. The compositions can optionally be in “ready-to-use” form, comprising two or more components to be mixed before use. The dyes produced can be mixed together to obtain predictable tones.

Said compositions, mixed in a suitable dilution with an activator, permanently dye the hair, and are consequently stable over time and withstand washing.

The compositions also possess improved sensitising power thanks to the absence of dyes which are classified, on the basis of their sensitising power, as “extreme” and thanks to the use of those which are classified as “strong”, “moderate” or “not classifiable”, as indicated in Opinion 1509/13 of the Scientific Committee on Consumer Safety (SCCS) entitled “Memorandum on hair dye chemical sensitisation”, dated Feb. 26, 2013.

The preferred primary dyes are N,N-bis(2-hydroxyethyl)-p-phenylene-diamine and p-aminophenol.

The preferred dye couplers are 4-chlororesorcinol, 2-methylresorcinol, 2,4-diaminophenoxyethanol.

The total quantity of the combination of primary dyes and couplers in the dye according to the invention preferably ranges between about 0.001 and 20% by weight, more preferably between about 0.002 and 10% by weight, and even more preferably between about 0.01 and 6.0% by weight.

When the dye is mixed with the activator, which in most cases is acidic (pH about 2 to 6.5), the pH of the ready-for-use dyes according to the invention acquires a value determined by the quantity of alkali in the dye and the quantity of acid in the oxidant, and by the mixing ratio. Depending on their composition, the ready-for-use dyes according to the invention can be weakly acidic, neutral or alkaline, and have a pH ranging from about 3 to 11, preferably from 6.5 to 11.

The composition can also include a pH adjuster selected, for example, from ammonia, monoethanolamine (MEA), 1-amino-2-propanol, 2-amino-2-methyl-propanol (AMP), 2-amino-2-methyl-1,3-propanediol, 2-amino-2-ethyl-1,3-propanediol and tris(hydroxymethyl)-aminomethane (tromethamine, Tris), sodium hydroxide, potassium hydroxide, urea, allantoin, arginine, tripotassium phosphate, sodium saccharine, triethanolamine (TEA) or combinations thereof.

The quantity of pH adjuster can range between 0.1 and 20% by weight, preferably between 0.2 and 10% by weight.

The alkanolamine can be present in free base form or salt form.

“Activator” means hydrogen peroxide, carbamide peroxide, perborates and persulphates or peracids. The preferred compound is hydrogen peroxide. The quantity can range from 0.1 to 50%.

The dyes can also contain one or more natural or synthetic additives, commonly used in solutions, creams, emulsions, gels, aerosols, foams, powders and granulates, for example solvents, such as water, low-molecular-weight aliphatic mono- or polyalcohols, esters and ethers thereof, for example alkanols, in particular having 1 to 4 carbon atoms, such as ethanol, n-propanol, isopropanol, butanol and isobutanol; bivalent or trivalent alcohols, in particular having 2 to 6 carbon atoms, such as ethylene glycol, propylene glycol, 1,3-propanediol, 1,4-butanediol, 1,5-pentanediol, 1,6-hexanediol, 1,2,6-hexanetriol, glycerin, diethylene glycol, dipropylene glycol, polyalkylene glycols, such as triethylene glycol, polyethylene glycol, tripropylene glycol and polypropylene glycol; low-molecular-weight alkyl ethers of multivalent alcohols, such as ethylene glycol monomethyl ether, ethylene glycol monoethyl ether, ethylene glycol monopropyl ether or ethylene glycol monobutyl ether, diethylene glycol monomethyl ether or diethylene glycol monoethyl ether, triethylene glycol monomethyl ether or triethylene glycol monoethyl ether; ketones and keto alcohols, in particular having 3 to 7 carbon atoms, such as acetone, methyl ethyl ketone, diethyl ketone, methyl isobutyl ketone, methyl phenyl ketone, cyclopentanone, cyclohexanone and diacetone alcohol; ethers such as dibutyl ether, tetrahydrofuran, dioxane or diisopropylether; esters such as ethyl formate, methyl formate, methyl acetate, ethyl acetate, propyl acetate, butyl acetate, phenyl acetate, ethylene glycol monoethyl ether acetate or acetic acid hydroxyethyl ester; amides such as N-methylpyrrolidone; and urea, tetramethyl urea and thiodiglycol; together with wetting agents or emulsifiers selected from anionic, cationic, non-ionogenic, amphoteric or zwitterionic agents, surfactants, such as fatty alcohol sulphates, alkylsulphonates, alkylbenzene sulphonates, alkylmethyl ammonium salts, alkylbetaine, a-olefin sulphonates, ethoxylated fatty alcohols, ethoxylated nonylphenols, fatty acid alkanolamines, ethoxylated esters of fatty acids, polyglycol ether sulphates of fatty acids and alkylpolyglycosides; thickeners, such as higher fatty alcohols, starch, cellulose derivatives, vaseline, paraffin oil, fatty acids and other fatty components in emulsified form, water-soluble polymer thickeners, such as natural gums, guar gum, xanthan gum, carob flour, pectin, dextran, agar-agar, amylose, amylopectin, dextrin, synthetic clays or hydrocolloids, such as polyvinyl alcohol; conditioning agents such as lanolin derivatives, cholesterol, pantothenic acid, water-soluble cationic polymers, protein derivatives, provitamins, vitamins, plant extracts, sugar and betaine; auxiliary agents such as electrolytes, antioxidants, fatty amides, sequestering agents, film-forming agents and preservatives, and beeswax.

The addition to the dyes of non-ionic and/or anionic surfactants or emulsifiers, such as fatty alcohol sulphates, in particular lauryl sulphate or sodium cocoyl sulphate; ethoxylated fatty alcohol sulphates, in particular sodium lauryl ether sulphates with 2 to 4 molecular units of ethylene oxide, ethoxylated esters of fatty acids, ethoxylated nonylphenols, ethoxylated fatty alcohols, alkylbenzene sulphonates or alkanolamides of fatty acids, in a total quantity preferably ranging from about 0.1 to 30% by weight, more preferably from 0.2 to 15% by weight, can be particularly advantageous in this case.

Examples of useful cationic surfactants are quaternary ammonium compounds; ammonium halides such as alkyltrimethylammonium chlorides, dialkyldimethylammonium chlorides and trialkylmethylammonium chlorides. Specific examples are cetyltrimethylammonium chloride, stearyltrimethyl-ammonium chloride, distearyldimethylammonium chloride, lauryldimethyl-ammonium chloride, lauryldimethylbenzylammonium chloride and tricetylmethyl-ammonium chloride. Other useful cationic surfactants are quatemised protein hydrolysates.

As well as non-ionic organic thickeners with properties similar to wax and non-ionic surfactants, the dye can include the usual cosmetic cationic resins. Particularly preferred are Polyquaternium-6 (poly(dimethyl-diallylammonium chloride)), Polyquaternium-7 (diethyldiallylammonium chloride/acrylamide copolymer), Polyquaternium-10 (cationic cellulose), Polyquaternium-11 (diethyl sulphate of N,N-dimethylaminoethylmethacrylic acid/PVP copolymer), Polyquaternium-22, Polyquaternium-35 and Polyquaternium-37 (trimethyl-aminoethyl methacrylate chloride polymer), either alone or in mixtures thereof. The total quantity of said cationic resins in the dye can range from about 0.1 to 6% by weight.

For the use of oxidative hair dyes, the dyes are mixed with an oxidative solution immediately before dyeing the hair, and a sufficient quantity of ready-for-use hair colouring mixture, generally about 60 to 200 grams depending on the thickness and quantity of the hair, is applied.

The mixture is left on the hair for 5 to 60 minutes at the temperature of 5 to 50° C., preferably for 35 minutes at 30° C.; the hair is then rinsed with water and dried. If necessary, the hair is washed with shampoo after rinsing and optionally rinsed again with a weak organic acid, such as an aqueous solution of tartaric acid. The hair is then dried.

The hair dye according to the invention gives a deep, protective, delicate hair colour. Due to improved colour balancing, all parts of the hair can be dyed, from undamaged roots to split ends.

The International Colour Chart (ICC) is a system used to classify hair dyes. This means that every dye has a code defining its colour. Said code can be used by manufacturers of colour charts or dyes. In practice, the ICC system uses numbers to define the depth (level) and tone of a given colour.

The colour level indicates how light or dark the shade is. The ICC system assigns a number to measure the level of lightness or darkness. Said values range from 1 to 10, wherein 1 denotes the darkest shade (black) and 10 the lightest shade (lightest blonde). Some manufacturers also use the codes 11 and 12 on their hair dyeing products to indicate platinum blonde shades. The usual level numbers and names are as follows:

| Level | Level name |
|---|---|
| 1 | Black |
| 2 | Very dark brown |
| 3 | Dark brown |
| 4 | Medium brown |
| 5 | Light brown |
| 6 | Dark blonde |
| 7 | Medium blonde |
| 8 | Light blonde |
| 9 | Very light blond |
| 10 | Lightest blond |
| 11 | Platinum blonde |

The tone indicates how cool or warm a colour is, and includes colours such as gold, ash and copper.

Although the level measurement is almost identical for all manufacturers, each manufacturer can vary the tone number at its discretion. Tone is indicated by a number, usually placed after the level, separated by a decimal point “.”, a comma “,” or a slash “/”. The classification used by the Applicant is set out below by way of example.

| Tone number | Tone name |
|---|---|
| 0 | Natural (grey-neutral) |
| 1 | Blue ash (blue) |
| 2 | Mauve ash (purple) |
| 3 | Gold (yellow) |
| 4 | Copper (orange) |
| 5 | Mahogany (violet red) |
| 6 | Red (red) |
| 7 | Khaki (green) |
| 8 | Pearl |

Some dyes can have a double tone, and it is usual to place two numbers after the decimal point of the level to express said characteristic. For example, if the colour chart contains the number 7.21, the first number indicates the medium blonde level (7), the second indicates the violet tone (2), and the third number indicates a secondary ash tone (3). Said colour will be called “violet-ash medium blonde.

The dyeing results obtained from the examples below, expressed in L*a*b* values, were measured with a Minolta Chroma Meter CR-200 colorimeter.

In the colour space L*a*b*, L* indicates sheen and a* and b* are the colour coordinates. a* and b* indicate the colour directions: +a* is the direction of red, −a* is the direction of green, +b* is the direction of yellow and −b* is the direction of blue.

Differences in colour can be expressed by the values AE, which are defined by the following equation:

ΔE=[(ΔL*)²+(Δa*)² +(Δb*)²]^{1/2}

### EXAMPLES

The ingredients listed in the examples are named according to the INCI nomenclature (European Community Decision 2006/257/EC as amended - International Nomenclature of Cosmetic Ingredients).

Table 1 shows the formula of the activators used for the examples below.

TABLE 1
Formulas A, B, C and D relate to the different
strengths of the activators.

| | A | B | C | D |
|---|---|---|---|---|
| INGREDIENTS | % | % | % | % |
| AQUA (WATER) | q.s. for | q.s. for | q.s. for | q.s. for |
| | 100 | 100 | 100 | 100 |
| HYDROGEN PEROXIDE | 12 | 9 | 6 | 3 |
| CETEARYL ALCOHOL | 3 | 3 | 3 | 3 |
| CETEARETH-20 | 0.6 | 0.6 | 0.6 | 0.6 |
| PHOSPHORIC ACID | 0.1 | 0.1 | 0.1 | 0.1 |
| SODIUM STANNATE | 0.2 | 0.2 | 0.2 | 0.2 |
| SODIUM LAURETH | 0.1 | 0.1 | 0.1 | 0.1 |
| SULPHATE | | | | |
| PROPYLENE GLYCOL | 0.1 | 0.1 | 0.1 | 0.1 |
| DISODIUM PYROPHOSPHATE | 0 | 0 | 0 | 0 |
| DIMETHICONE | 0.1 | 0.1 | 0.1 | 0.1 |
| PEG-40 CASTOR OIL | 0.1 | 0.1 | 0.1 | 0.1 |
| PENTASODIUM PENTETATE | 0.1 | 0.1 | 0.1 | 0.1 |
| ETIDRONIC ACID | 0.1 | 0.1 | 0.1 | 0.1 |
| C12-13 ALKYL LACTATE | 1 | 1 | 1 | 1 |

TABLE 2
Formulas “F” and “I” are formulas according
to the invention, while formulas “E”, “G”
and “H” are comparative examples.

| | E | F | G | H | I |
|---|---|---|---|---|---|
| | shade | shade | shade | shade | shade |
| | 7.0 | 7.0 | 7.0 | 7.3 | 7.3 |
| INCI | % | % | % | % | % |
| AQUA | q.s. for | q.s. for | q.s. for | q.s. for | q.s. for |
| | 100 | 100 | 100 | 100 | 100 |
| STEARYL ALCOHOL | 6.00 | 6.00 | 6.00 | 6.00 | 6.00 |
| CETEARYL ALCOHOL | 4.00 | 4.00 | 4.00 | 4.00 | 4.00 |
| CETEARETH-50 | 4.00 | 4.00 | 4.00 | 4.00 | 4.00 |
| AMMONIA | 1.50 | 1.50 | 1.50 | 1.50 | 1.50 |
| PROPYLENE GLYCOL | 5.00 | 5.00 | 5.00 | 5.00 | 5.00 |
| LAURYL ALCOHOL | 2.00 | 2.00 | 2.00 | 2.00 | 2.00 |
| MYRISTYL ALCOHOL | 1.00 | 1.00 | 1.00 | 1.00 | 1.00 |
| COCAMIDOPROPYL | 1.00 | 1.00 | 1.00 | 1.00 | 1.00 |
| BETAINE | | | | | |
| CETEARETH-50 | 3.00 | 3.00 | 3.00 | 3.00 | 3.00 |
| PEG-40 HYDROGE- | 2.00 | 2.00 | 2.00 | 2.00 | 2.00 |
| NATED CASTOR OIL | | | | | |
| ETHANOLAMINE | 1.00 | 1.00 | 1.00 | 1.00 | 1.00 |
| PARFUM | 1.00 | 1.00 | 1.00 | 1.00 | 1.00 |
| POLYQUATERNIUM- | 0.50 | 0.50 | 0.50 | 0.50 | 0.50 |
| 22 | | | | | |
| DECYLTETRA- | 0.80 | 0.80 | 0.80 | 0.80 | 0.80 |
| DECANOL | | | | | |
| SODIUM SULPHITE | 0.40 | 0.40 | 0.40 | 0.40 | 0.40 |
| ERYTHORBIC ACID | 0.10 | 0.10 | 0.10 | 0.10 | 0.10 |
| EDTA | 0.10 | 0.10 | 0.10 | 0.10 | 0.10 |
| BISABOLOL | 0.10 | 0.10 | 0.10 | 0.10 | 0.10 |
| OLETH-5 PHOSPHATE | 1.00 | 1.00 | 1.00 | 1.00 | 1.00 |
| DIOLEYL PHOSPHATE | 1.00 | 1.00 | 1.00 | 1.00 | 1.00 |
| DIMETH- | 0.10 | 0.10 | 0.10 | 0.10 | 0.10 |
| YLPABAMIDOPRO- | | | | | |
| PYL LAURDIMONIUM | | | | | |
| TOSYLATE | | | | | |
| SODIUM HYALURO- | 0.50 | 0.50 | 0.50 | 0.50 | 0.50 |
| NATE CROSSPOLY- | | | | | |
| MER | | | | | |
| PENTYLENE GLYCOL | 0.50 | 0.50 | 0.50 | 0.50 | 0.50 |
| GLYCERIN | 1.00 | 1.00 | 1.00 | 1.00 | 1.00 |
| TOLUENE-2,5-DI- | 1.20 | | | 0.8 | |
| AMINE SULPHATE | | | | | |
| RESORCINOL | 0.4 | | | 0.2 | |
| 2-METHYLRESOR- | 0.2 | 0.8 | 0.8 | 0.65 | 1.00 |
| CINOL | | | | | |
| m-AMINOPHENOL | 0.1 | | 0.1 | 0.1 | |
| 2.4-DIAMINOPHE- | 0.01 | 0.01 | | | |
| NOXYETHANOL HCl | | | | | |
| N.N-BIS(2-HYDROXY- | | 1.2 | 1.2 | | 0.7 |
| ETHYL)-p-PHENYL- | | | | | |
| ENEDIAMINE | | | | | |
| SULPHATE | | | | | |
| p-AMINOPHENOL | | 0.5 | 0.5 | 0.6 | 0.7 |
| 2-AMINO-3-HY- | 0.02 | 0.02 | | 0.06 | 0.06 |
| DROXYPYRIDINE | | | | | |
| 4-CHLORORESORCIN- | | 0.2 | | | 0.05 |
| OL | | | | | |

In Table 3 the following values have been assigned on the basis of their sensitising power reported in the “Memorandum on hair dye chemical sensitisation” dated 26 Feb. 2013.

0: EXTREME

1: STRONG

2: MODERATE

NC: NOT CLASSIFIED

On the basis of the dyes contained in the dye compositions of formulas E, F, G, H and I, the sensitisation indexes were calculated with the following formula:

Sum of individual sensitisation indexes/number of dyes present in the composition.

On the basis of the sensitisation index it can be stated that a higher value indicates improved sensitising power (as also stated in Opinion 1509/13 of the Scientific Committee on Consumer Safety (SCCS) relating to evaluation of the sensitising power of the dyes evaluated by the SCCS).

Dyes considered as NC were not taken into consideration when calculating the index.

TABLE 3
Sensitisation index (SI)

| INCI | E | F | G | H | I |
|---|---|---|---|---|---|
| TOLUENE-2,5-DIAMINE | 0 | | | 0 | |
| SULPHATE | | | | | |
| RESORCINOL | 1 | | | 1 | |
| 2-METHYLRESORCINOL | 2 | 2 | 2 | 2 | 2 |
| m-AMINOPHENOL | 1 | | 1 | 1 | |
| 2.4-DIAMINOPHENOL HCl | 2 | 2 | | | |
| N.N-BIS(2-HYDROXYETHYL)-p- | | 1 | 1 | | 1 |
| PHENYLENEDIAMINE | | | | | |
| SULPHATE | | | | | |
| p-AMINOPHENOL | | 1 | 1 | 1 | 1 |
| 2-AMINO-3-HYDROXYPYRIDINE | nc | nc | | nc | nc |
| 4-CHLORORESORCINOL | | 2 | | | 2 |
| SI: Ratio between sum of values/ | 1.20 | 1.60 | 1.25 | 1.00 | 1.50 |
| number of dyes | | | | | |

As shown in Table 3, the combinations according to the invention possess a higher sensitisation index value than the reference combinations. In particular, composition F has a higher value than the same composition “E” formulated with p-toluenediamine (Toluene-2,5-Diamine Sulphate). Composition F also has a higher SI value than composition G, which contains the dye m-aminophenol.

Composition “I” according to the invention must be compared with composition “H” wherein, once again, higher SI values are obtained by excluding p-toluenediamine and m-aminophenol from the composition.

In Tables 4 and 5, locks of grey hair dyed with compositions “E”, “F”, “G”, “H” and “I” were read with a Minolta Chroma Meter CR-200 colorimeter.

The compositions were mixed with the activator of composition “B” in the ratio 1:1.5 (20 g of dye and 30 g of activator) and left to act for 35 minutes at 29° C.

ΔE values below 1 are considered to be insignificant differences, whereas values exceeding 1 are significantly different. As shown by the ΔE data in Table 4, compositions F and G are in line with composition E, while Table 5 demonstrates that composition I according to the invention gives the same colour as reference composition H.

TABLE 4
Colorimeter values of lock of grey hair after
dyeing with compositions E, F and G.

| | E shade 7 | F shade 7 | G shade 7 |
|---|---|---|---|
| L* | 20.93 | 21.05 | 21.21 |
| a* | 3.41 | 3.07 | 3.01 |
| b* | 3.83 | 3.95 | 3.09 |
| dE | Target | 0.38 | 0.88 |

TABLE 5
Colorimeter values of lock of grey hair
after dyeing with compositions H and I.

| | H shade 7.3 | I shade 7.3 |
|---|---|---|
| L* | 22.90 | 23.5 |
| a* | 6.53 | 6.2 |
| b* | 9.56 | 10.01 |
| dE | Target 7.3 | 0.80 |

When composition E is mixed with 50% H (mixture L) and activator “B” is added, maintaining a dilution ratio of 1:1.5 and leaving the substance to act for 35 minutes at 29° C., the colorimeter values indicated in Table 6 are obtained. The values for mixtures “M” and “N” are also reported in Table 6. Mixture M is the mixture according to the invention. In this example, a natural shade (7.0) is mixed with a gold shade of the same level (7.3); the result to be expected is as reported for mixture L, or with higher values of parameter b*. However, value a* must remained as similar as possible to mixture L.

TABLE 6
Colorimeter values of lock of grey hair after dyeing with
the compositions mixed in a 1:1 ratio and then further
mixed with a quantity of activator B in a 1:1.5 ratio.

| | L | M | N |
|---|---|---|---|
| | 50% E + 50% H | 50% F + 50% I | 50% G + 50% I |
| L* | 20.66 | 21.45 | 21.56 |
| a* | 5.45 | 4.97 | 3.21 |
| b* | 7.68 | 8.42 | 6.35 |

As shown in Table 6, mixture M according to the invention gives the best results in terms of b*, while maintaining the values of a* almost unchanged. Mixture “N”, which contains m-aminophenol, produces a significant reduction in b* and a* which corresponds to a cooler colour than that given by mixture “L”, confirming that m-aminophenol does not produce predictable colours.

Table 7 sets out some examples according to the invention with the corresponding SI compared with the prior art. The compositions according to the invention are Q, T, Z, AC, AF, AI, AN, AQ, AT and AZ. 1-naphthol, which is classified as a “Strong” sensitiser, is also included in some examples.

TABLE 7

| | O shade | P shade | Q shade | R shade | S shade | T shade |
|---|---|---|---|---|---|---|
| Composition/INCI | 1.0 | 1.0 | 1.0 | 5.0 | 5.0 | 5.0 |
| RESORCINOL | 1 | 1 | | 1 | 1 | |
| 2-METHYLRESORCINOL | 2 | 2 | | 2 | 2 | 2 |
| 2.4-DIAMINOPHENOXY- | 2 | 2 | 2 | 2 | 2 | 2 |
| ETHANOL HCl | | | | | | |
| N.N-BIS(2-HYDROXYETHYL)-p- | | | 1 | | | 1 |
| PHENYLENEDIAMINE SULPHATE | | | | | | |
| p-AMINOPHENOL | | | 1 | | | 1 |
| 2-AMINO-3-HYDROXYPYRIDINE | | | | | | |
| 4-CHLORORESORCINOL | | | 2 | | | 2 |
| m-AMINOPHENOL | 1 | 1 | | 1 | 1 | |
| 1-HYDROXYETHYL 4,5-DIAMINO | 1 | 1 | | | | |
| PYRAZOLE SULPHATE | | | | | | |
| p-PHENYLENEDIAMINE | 0 | | | 0 | | |
| TOLUENE-2,5-DIAMINE SULPHATE | | 0 | | | 0 | |
| 1-NAPHTHOL | | | | | | |
| SI: Ratio between | 1.17 | 1.17 | 1.50 | 1.20 | 1.20 | 1.60 |
| sum of values/number of dyes | | | | | | |

| | U shade | V shade | Z shade | AA shade | AB shade | AC shade |
|---|---|---|---|---|---|---|
| Composition/INCI | 10.0 | 10.0 | 10.0 | 11.0 | 11.0 | 11.0 |
| RESORCINOL | 1 | 1 | | | | |
| 2-METHYLRESORCINOL | 2 | 2 | 2 | 2 | 2 | |
| 2.4-DIAMINOPHENOXY-ETHANOL HCl | 2 | 2 | 2 | 2 | 2 | 2 |
| N.N-BIS(2-HYDROXYETHYL)-p- | | | 1 | | | 1 |
| PHENYLENEDIAMINE SULPHATE | | | | | | |
| p-AMINOPHENOL | 1 | 1 | 1 | | | |
| 2-AMINO-3-HYDROXYPYRIDINE | | | | | | |
| 4-CHLORORESORCINOL | | | 2 | | | |
| m-AMINOPHENOL | 1 | 1 | | 1 | 1 | 1 |
| 1-HYDROXYETHYL 4,5-DIAMINO | | | | | | |
| PYRAZOLE SULPHATE | | | | | | |
| p-PHENYLENEDIAMINE | 0 | | | 0 | | |
| TOLUENE-2,5-DIAMINE SULPHATE | | 0 | | | 0 | |
| 1-NAPHTHOL | | | | | | |
| SI: Ratio between | 1.17 | 1.17 | 1.60 | 1.25 | 1.25 | 1.33 |
| sum of values/number of dyes | | | | | | |

| | AD shade | AE shade | AF shade | AG shade | AH shade | AI shade |
|---|---|---|---|---|---|---|
| Composition/INCI | 5.1 | 5.1 | 5.1 | 5.2 | 5.2 | 5.2 |
| RESORCINOL | 1 | 1 | | | | |
| 2-METHYLRESORCINOL | 2 | 2 | 2 | | | |
| 2.4-DIAMINOPHENOXY-ETHANOL HCl | 2 | 2 | 2 | 2 | 2 | 2 |
| N.N-BIS(2-HYDROXYETHYL)-p- | | | 1 | | | 1 |
| PHENYLENEDIAMINE SULPHATE | | | | | | |
| p-AMINOPHENOL | | | 1 | 1 | 1 | 1 |
| 2-AMINO-3-HYDROXYPYRIDINE | | | | | | |
| 4-CHLORORESORCINOL | | | 2 | | | 2 |
| m-AMINOPHENOL | 1 | 1 | | | | |
| 1-HYDROXYETHYL 4,5-DIAMINO | | | | 1 | 1 | |
| PYRAZOLE SULPHATE | | | | | | |
| p-PHENYLENEDIAMINE | 0 | | | 0 | | |
| TOLUENE-2,5-DIAMINE SULPHATE | | 0 | | | 0 | |
| 1-NAPHTHOL | | | | 1 | 1 | |
| SI: Ratio between | 1.20 | 1.20 | 1.60 | 1.00 | 1.00 | 1.50 |
| sum of values/number of dyes | | | | | | |
| | AL shade | AM shade | AN shade | AO shade | AP shade | AQ shade |
| Composition/INCI | 5.3 | 5.3 | 5.3 | 5.4 | 5.4 | 5.4 |
| RESORCINOL | 1 | 1 | | 1 | 1 | |
| 2-METHYLRESORCINOL | 2 | 2 | 2 | 2 | 2 | |
| 2.4-DIAMINOPHENOXY-ETHANOL HCl | 2 | 2 | 2 | | | 2 |
| N.N-BIS(2-HYDROXYETHYL)-p- | | | 1 | | | |
| PHENYLENEDIAMINE SULPHATE | | | | | | |
| p-AMINOPHENOL | 1 | 1 | 1 | 1 | 1 | 1 |
| 2-AMINO-3-HYDROXYPYRIDINE | | | | nc | nc | nc |
| 4-CHLORORESORCINOL | | | | | | 2 |
| m-AMINOPHENOL | 1 | 1 | | 1 | 1 | |
| 1-HYDROXYETHYL 4,5-DIAMINO | | | | | | |
| PYRAZOLE SULPHATE | | | | | | |
| p-PHENYLENEDIAMINE | 0 | | | 0 | | |
| TOLUENE-2,5-DIAMINE SULPHATE | | 0 | | | 0 | |
| 1-NAPHTHOL | | | | | | |
| SI: Ratio between | 1.17 | 1.17 | 1.50 | 1.00 | 1.00 | 1.67 |
| sum of values/number of dyes | | | | | | |
| | AR shade | AS shade | AT shade | AU shade | AV shade | AZ shade |
| INCI | 5.5 | 5.5 | 5.5 | 5.6 | 5.6 | 5.6 |
| RESORCINOL | 1 | 1 | | | | |
| 2-METHYLRESORCINOL | | | | | | |
| 2.4-DIAMINOPHENOXY-ETHANOL HCl | | | 2 | | | |
| N.N-BIS(2-HYDROXYETHYL)-p- | | | 1 | | | 1 |
| PHENYLENEDIAMINE SULPHATE | | | | | | |
| p-AMINOPHENOL | 1 | 1 | 1 | 1 | 1 | 1 |
| 2-AMINO-3-HYDROXYPYRIDINE | nc | nc | nc | | | |
| 4-CHLORORESORCINOL | | | 2 | | | |
| m-AMINOPHENOL | 1 | 1 | | 1 | 1 | |
| 1-HYDROXYETHYL 4,5-DIAMINO | | | | 1 | 1 | 1 |
| PYRAZOLE SULPHATE | | | | | | |
| p-PHENYLENEDIAMINE | 0 | | | 0 | | |
| TOLUENE-2,5-DIAMINE SULPHATE | | 0 | | | 0 | |
| 1-NAPHTHOL | | | | | | |
| SI: Ratio between | 0.75 | 0.75 | 1.50 | 0.75 | 0.75 | 1.00 |
| sum of values/number of dyes | | | | | | |

1. A method for reducing the sensitising effect of oxidative hair-dyeing compositions applied to the scalp of subjects undergoing hair-dyeing treatments, said method comprising:
applying to the scalp a composition containing an activator, at least one primary dye selected from N,N-bis(2-hydroxyethyl)-p-phenylenediamine, p-aminophenol, 4-amino-m-cresol, hydroxyethyl-p-phenylenediamine and 1-hydroxyethyl-4,5-diamino pyrazole and at least one dye coupler selected from 4-chlororesorcinol, 2-methylresorcinol, 2,4-diaminophenoxyethanol and 2-amino-3-hydroxypyridine,
wherein said compositions do not contain p-phenylenediamine, p-toluenediamine, m-aminophenol or resorcinol.

2. A method according to claim 1 wherein the compositions are in the form of O/W (oil-in-water) or W/O (water-in-oil) emulsions, liquids, biphasic liquids, gels, oils, creams or foams.

3. A method according to claim 1 wherein the compositions are in “ready to use” form, comprising two or more components to be mixed before use.

4. A method according to claim 1 wherein the compositions further comprise pH adjusters, activators, surfactants, emulsifiers or thickeners.

5. A method according to claim 1 wherein the activator is hydrogen peroxide.

6. A method according to claim 1 wherein the primary dye and the dye coupler are present in the compositions, in combination, in amounts ranging from 0.01 to 4.0% by weight of the total weight of the composition.

7. A method according to claim 1 wherein the compositions have a pH ranging from 6.5 to 11.

8. A method according to claim 1 comprising the steps of:
a. leaving the composition on the hair for a time ranging from 10 to 45 minutes at 20-40° C.;
b. optionally using heat sources during phase (ii);
c. washing hair with water;
d. optionally contacting hair with a towel and drying.

9. Method according to claim 1 wherein two or more dyes are applied, in order to obtain consistent color and tones.

10. An oxidative hair dyeing composition containing an activator, at least one primary dye selected from N,N-bis(2-hydroxyethyl)-p-phenylenediamine p-aminophenol, hydroxyethyl-p-phenylenediamine and 4-amino-m-cresol and at least one dye coupler selected from 4-chlororesorcinol, 2-methylresorcinol and 2,4-diaminophenoxyethanol, wherein said compositions do not contain p-phenylenediamine, p-toluenediamine, m-aminophenol or resorcinol.
